(19) United States
(12) Patent Application Publication
(10) Pub. No.: US 2024/0308944 A1
(43) Pub. Date: Sep. 19, 2024

(54) METHOD FOR THE MANUFACTURE OF BISPHENOL A

(71) Applicant: SABIC Global Technologies B.V., Bergen op Zoom (NL)

(72) Inventors: Surya Prakasa Rao Daliparthi, Bangalore (IN); Paulus Johannes Maria Eijsbouts, Nieuwkuijk (NL); Gert-Jan Schoenmakers, Prinsenbeek (NL)

(21) Appl. No.: 18/576,586

(22) PCT Filed: Jun. 13, 2022
(86) PCT No.: PCT/EP2022/065944

§ 371 Date: Jan. 4, 2024

(30) Foreign Application Priority Data
Jul. 5, 2021 (EP) 21183693.7

(51) Int. Cl. C07C 37/84; B01J 14/00; B01J 31/08; C07C 37/74

(52) U.S. Cl. CPC C07C 37/84; B01J 14/005; B01J 31/08; C07C 37/74; B01J 2219/00033; C07C 2601/16

(57) ABSTRACT

A method for the continuous manufacture of bisphenol A comprising i) feeding one or more feed streams comprising phenol and acetone to a reactor and reacting said acetone and phenol in the presence of an ion-exchange resin catalyst thereby forming a product stream comprising bisphenol A, phenol, acetone, water and by-products, ii) crystallising bisphenol A and/or bisphenol A/phenol adduct crystals from said product stream in one or more crystallisation units thereby forming a slurry consisting of said crystals and a mother liquor, iii) separating the mother liquor from the crystals in one or more solid-liquid separation units, iv) heating the mother liquor in a heat recovery unit and feeding the heated mother liquor to a distillation column, v) separating the mother liquor in the distillation column thereby forming a bottom stream comprising phenol and a top stream comprising water, vi) cooling the bottom stream in said heat recovery unit.

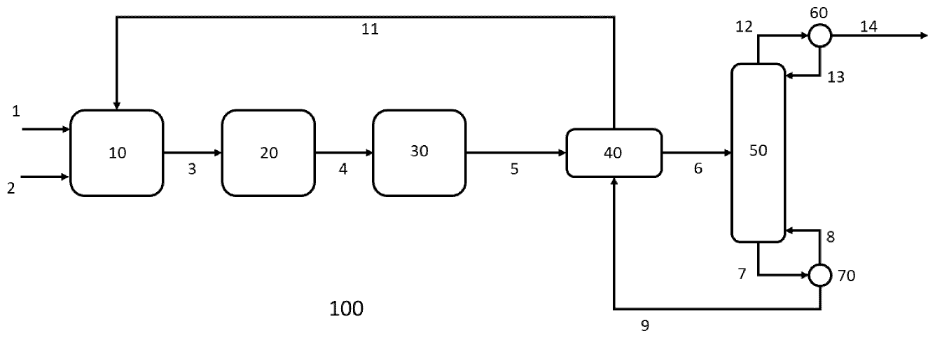

### CROSS-REFERENCE TO RELATED APPLICATIONS

This application is a National Stage application of PCT/EP2022/065944, filed Jun. 13, 2022, which claims the benefit of European Application No. 21183693.7, filed Jul. 5, 2021, both of which are incorporated by reference in their entireties herein.

### BACKGROUND

The present invention relates to a method for the manufacture of bisphenol A.

Bisphenol A (2,2′-bis(4-hydroxyphenyl)propane, also known as p,p-BPA) is predominantly used as an intermediate for the production of other products. Bisphenol A is used inter alia for the production of polycarbonate resins, epoxy resins, unsaturated polyester, polysulphone, polyetherimide and polyarylate resins.

Bisphenol A is commercially prepared by condensing two moles of phenol with one mole of acetone in the presence of an acid catalyst as shown in the equation below.

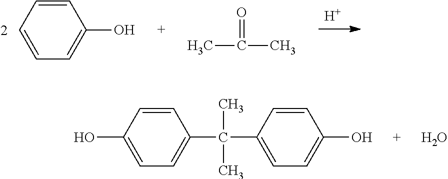

The reaction is usually carried in a continuous manner by passing the reactants, either in upflow or in down-flow through a bed of ion-exchange resin catalyst. After that the resulting product mixture is subjected to adduct crystallisation wherein bisphenol A/phenol adduct crystals are formed. The so obtained slurry is then subjected to a solid-liquid separation which optionally includes the addition of fresh phenol. This separation results in a stream of adduct crystals and a stream of what is often referred to as mother liquor. In order to improve the selectivity of the reaction and for reasons of efficiency more in general it was found that recycling the mother liquor to the reactor is advantageous. However it was also found that the water that is contained in the mother liquor reduces the activity of the catalyst and accordingly needs to be removed. This is known per se.

US 2005/0177007 discloses a process for producing bisphenol A comprising (a) reacting phenol with acetone in the presence of acidic catalyst to form a reaction mixture containing bisphenol A and water, b) removing the water by distillation in a distillation column, and obtaining a bottom product, c) separating bisphenol-A/phenol adduct crystals from the reaction mixture by crystallization and filtration, wherein the bottom temperature of the column is 100 to 150 C, the overhead temperature of the column is 20 to 80 C, the absolute pressure is 50 to 300 mbar at the head of the column and 100 to 300 mbar at the bottom of the column, and wherein said (c) is carried out before or after said (b).

U.S. Pat. No. 6,635,788 discloses a method for the manufacture of bisphenols comprising: introducing a combined feed stream comprising a feed stream and a recycle stream into a reactor system comprising at least one reactor containing a catalytic proportion of an acid catalyst and wherein the combined feed stream comprises a carbonyl compound and a stoichiometric excess of phenol; removing from the reactor system a reactor effluent; splitting the reactor effluent into a crystallization feed stream and an effluent recycle stream; extracting from said crystallization feed stream a bisphenol adduct, remainder comprising a mother liquor stream, dehydrating said mother liquor stream with and said effluent recycle stream in a dehydrator wherein excess water and carbonyl compound are removed; and recycling the dehydrated mother liquor and the dehydrated effluent recycle stream back to the combined feed Stream to effect improved production of p,p-bisphenol, along with increased reactor selectivity and reduced promoter quantities.

### SUMMARY

It is an object of the present invention to provide for a more energy efficient process for the manufacture of bisphenol A.

To that extent the present invention is directed at a method for the continuous manufacture of bisphenol A comprising
feeding one or more feed streams comprising phenol and acetone to a reactor and reacting said acetone and phenol in the presence of an ion-exchange resin catalyst thereby forming a product stream comprising bisphenol A, phenol, acetone, water and by-products,
crystallising bisphenol A and/or bisphenol A/phenol adduct crystals from said product stream in one or more crystallisation units thereby forming a slurry consisting of said crystals and a mother liquor,
separating the mother liquor from the crystals in one or more solid-liquid separation units,
heating the mother liquor in a heat recovery unit and feeding the heated mother liquor to a distillation column,
separating the mother liquor in the distillation column thereby forming a bottom stream comprising phenol and optionally a minor amount of water and a top stream comprising water and optionally a minor amount of phenol,
cooling the bottom stream in said heat recovery unit.

More in particular the present invention relates to a method for the continuous manufacture of bisphenol A comprising
feeding one or more feed streams comprising phenol and acetone to a reactor and reacting said acetone and phenol in the presence of an ion-exchange resin catalyst thereby forming a product stream comprising bisphenol A, phenol, acetone, water and by-products,
crystallising bisphenol A and/or bisphenol A/phenol adduct crystals from said product stream in one or more crystallisation units thereby forming a slurry consisting of said crystals and a mother liquor,
separating the mother liquor from the crystals in one or more solid-liquid separation units,
heating the mother liquor in a heat recovery unit and feeding the heated mother liquor to a distillation column,
separating the mother liquor in the distillation column thereby forming a bottom stream comprising phenol and optionally a minor amount of water and a top stream comprising water and optionally a minor amount of phenol,
cooling the bottom stream in said heat recovery unit,
   wherein in the heat recovery unit heat is exchanged between the bottom stream and the mother liquor.

By exchanging the heat between the bottom stream and the mother liquor the need for additional heating and/or cooling energy and/or means is reduced to a minimum so that accordingly the object of the invention is met at least in part.

### BRIEF DESCRIPTION OF THE DRAWINGS

The present invention will now be further elucidated on the basis of the appended FIGURE which is no way to be regarded as limiting the present invention.

In the FIGURE at least part of an integrated process for the manufacture of bisphenol A is schematically shown.

### DETAILED DESCRIPTION

Phenol and acetone are fed to reactor 10 by means of feed streams 1 and 2 respectively.

The phenol stream 1 may consist of fresh phenol coming from an on-site phenol manufacturing unit or from an external source. The phenol stream 1 may also comprise a mixture of fresh phenol and recycled phenol. For example any phenol present in top stream 12 may be isolated, purified and combined with phenol stream 1.

The acetone stream 2 may consist of fresh acetone coming from an on-site acetone manufacturing unit or from an external source. The acetone stream 2 may also comprise a mixture of fresh acetone and recycled acetone. For example any acetone present in top stream 12 may be isolated, purified and combined with acetone stream 2.

In reactor 10 the phenol and acetone are reacted in the presence of an ion-exchange resin catalyst resulting in a product stream 3. Reactor 10 may operate in a down-flow or an upflow mode as is known to a skilled person wherein the ion-exchange resin catalyst is preferably present as a fixed bed. The reaction temperature can be from 40 to 150° C., preferably from 60 to 110° C., more preferably from 50 to 100° C. If the reaction temperature is lower than 40° C., not only the reaction speed is slow but also the reaction solution has a very high viscosity and may solidify. On the other hand, if the reaction temperature exceeds 150° C., it becomes difficult to control the reaction, and the selectivity of bisphenol A (p,p BPA) is lowered. In addition, the catalyst may be decomposed or deteriorated.

The catalyst is an acidic catalyst, such as sulphonic acid type ion exchange resin. Ion-exchange resins are well known in the art and described in various sources, such as Kirk-Othmer's Encyclopedia of Chemical Technology, 3rd Edition, Vol. 9, pp. 256 and 296-297 (1980); and Vol. 13, pp. 678 et seq. (1981). The use of ion-exchange resins for catalysis is also described in Kirk-Othmer's Encyclopedia of Chemical Technology, 4th Edition, Vol. 14, pp. 777-778 (1995). In particular, strong-acid types of resins are suitably employed. Preferably, the catalysts are sulfonated aromatic resins comprising hydrocarbon polymers having a plurality of pendant sulfonic acid groups. The pendant sulfonic acid groups are typically 2 or 4% divinyl benzene cross-linked. Examples include sulphonated styrene divinylbenzene copolymers, sulphonated cross linked styrene polymers, phenol formaldehyde sulphonic acid resins, and benzene formaldehyde sulphonic acid resins. These catalysts may be used individually or in combination.

The catalyst preferably comprises a chemically attached promotor for improving the reaction selectivity towards the formation of p,p-bisphenol A. The use of an attached promotor avoids the need for a separate, generally sulfur containing, promotor which requires isolation and further processing downstream of the reactor.

The phenol to acetone ratio is preferably from 40 to 5, preferably from 30 to 10, more preferably from 25 to 10, wherein the ratio is based on the weight of the phenol and acetone that is added to the reactor.

During the reaction bisphenol A (i.e. p,p-bisphenol A) is formed as the main and desired reaction product. However, other isomers or byproducts may also be formed which include in particular o,p-bisphenol A, 3-(4-hydroxyphenyl)-1,1,3-trimethyl-2H-inden-5-ol (“cyclic dimer 1”); 2,4-bis[1-(4-hydroxyphenyl)isopropyl]phenol (“BPX 1”); 4-(2,2,4-trimethylchroman-4-yl)phenol (“chroman 1”); 4-(2,4,4-trimethyl-3,4-dihydro-2H-chromen-2-yl)phenol (“chroman 1.5”); 1,1′-spirobi[1H-indene]-6,6′-diol,2,2′,3,3′-tetrahydro-3,3,3′,3′-tetramethyl (“spirobiindane”).

Typically the product stream comprises
from 10 to 40, preferably 15 to 35, more preferably from 20 to 30 wt. % bisphenol A,
from 55 to 75 wt. %, preferably from 60 to 70 wt. % phenol,
from 0 to 3 wt. %, preferably 0.01 to 1.0 wt. % of acetone,
from 2 to 20 wt. %, preferably from 5 to 15 wt. % by-products,
from 0 to 5 wt. %, preferably 1 to 3 wt. % water.
   For the avoidance of doubt, the total of the product stream equals 100 wt. %.

More preferably the product stream comprises
from 23 to 25 wt. % of bisphenol A
from 64 to 68 wt. % of phenol
from 0 to 0.5 wt. % of acetone
from 7 to 9 wt. % of by-products
from 1 to 3 wt. % of water.
   For the avoidance of doubt, the total of the product stream equals 100 wt. %.

From the reactor a product stream 3 is then introduced into a crystallisation unit 20 so as to form a slurry consisting of bisphenol A and/or bisphenol A/phenol adduct crystals and a mother liquor. It is noted that prior to crystallisation unit 20 product stream 3 may be split such that only part of the product stream is introduced in crystallisation unit 20. The term mother liquor is to be understood as being a stream of mother liquor. Thus, while reference is generally made to mother liquor this term is interchangeable with the term mother liquid stream.

In addition to product stream 3 further streams may be introduced into crystallisation unit 20, such as for example a stream comprising a solvent intended to support a desired crystallisation process or a desired crystal form. For example, in case crystals of bisphenol A would be desired then it may be convenient to add a co-solvent as a result of which the formation of bisphenol A crystals is favoured over the formation of bisphenol A/phenol adduct crystals. Such solvent may be one or more selected from the group consisting of toluene, benzene, xylene, hexane, heptane, trichloro-ethylene, and dichloro-methylene, preferably toluene. In an aspect of the invention a further stream introduced into the one or more crystallisation units may comprise or consist of phenol.

The crystallisation unit 20 may comprise one or more crystallisers in series or may comprise two or more crystallisation lines operated in parallel with each crystallisation line comprising one or more crystallisers. The conditions for crystallising bisphenol A and in particular bisphenol A/phenol adducts are known to the skilled person.

From crystallisation unit 20 a slurry consisting of said crystals and a mother liquor is obtained and (continuously) introduced to one or more solid/liquid separation units 30. The appended FIGURE only shows a single such unit, but multiple units 30 may be operated in parallel. An example of a solid-liquid separation unit is a rotating vacuum filter comprising a filter drum with a perforated sector on a lateral surface of the filter drum and a filter, such as for example a filter cloth attached to the filter drum and covering said perforated sector.

A vacuum pump is in fluid communication with an interior surface of the perforated sector. The slurry 4 is sprayed on the filter while the mother liquor is sucked into the interior of the filter drum and transported for further processing as mother liquor 5. A stream of crystals (not shown) is obtained by scraping of the layer of crystals on the filter cloth. A suitable filter drum is disclosed in WO 2018/225014. In order to wash the crystals an amount of fresh phenol may be used. This phenol thereby becomes part of the mother liquor stream 5. Accordingly, during the separating of the mother liquor from the crystals in the one or more solid-liquid separation units additional phenol is added to the mother liquor resulting from said washing of the crystals. The temperature of mother liquor stream when leaving the separation unit is from 40 to 75° C., preferably 45 to 65° C. The bottom stream typically comprises from 75 to 85 wt. % of phenol and from 0 to 0.3, such as 0.10 to 0.25 wt. % of water, based on the weight of the bottom stream. The balance consists of BPA and by-products, Typically the bottom stream further comprise from 8 to 12 wt. % of p,p-BPA, from 2 to 4 wt. % of o,p-BPA, at most 2 wt. % of BPX-1 and at most 0.5 wt. % of BPX-2 (4-(2-(4-(4-hydroxyphenyl)-2,2,4 trimethylchroman-6 yl)propan-2-yl)phenol.

Mother liquor stream 5 is introduced into heat recovery unit 40 wherein its temperature is increased to a temperature from 85 to 110, preferably from 90 to 105° C. The heat that is required for the increase in temperature originates at least in part from the bottom stream 9 coming from distillation column 50. Depending on how the plant is operated preferably at least 50%, more preferably at least 65, even more preferably at least 85% of the required heat comes from cooling of stream 9. It is most preferred that no additional heat is required, i.e. that 100% of the energy needed to heat mother liquor stream 5 comes from the cooling of stream 9. Similarly it is preferred that no additional cooling energy is required.

Heat recovery unit 40 may comprise one or more heat exchangers wherein the heat is exchanged between mother liquor stream 5 and distillation column bottom stream 9. For example known shell and tube heat exchangers may be used wherein the mother liquor is passed through the shell and the bottom stream 9 through the tubes or vice versa. It is also possible that in a first heat exchanging device energy from bottom stream 9 is transported to a heating medium, such as for example a fluid like oil or water and wherein the heating medium is consecutively used to provide heat to the mother liquor stream. In order to be able to provide sufficient cooling and/or sufficient heating further cooling or heating means (not shown) may be comprised in heat recovery unit 40. Heat exchangers for this purpose are known per se to the skilled person.

For the purpose of providing additional heating and/or cooling capacity the heat recovery unit may comprises additional heating or cooling units that rely on external energy being provided or withdrawn therefrom. Apart from providing said additional heating or cooling capacity this also allows the heat recovery unit to better deal with variations in the composition and/or flow rates of the streams being fed to the heat recovery unit.

From the heat recovery unit the heated mother liquor stream 6 is introduced into distillation column 50 where the mother liquor is separated into a water rich stream top stream 12 and a phenol rich bottom stream 7.

Column 50 preferably comprises n stages wherein the mother liquor is introduced between stage n-5 and n-2, wherein n is from 7 to 13, preferably from 9 to 11.

Distillation column 50 preferably comprises a condenser 60 for condensing at least part of the column top stream 12 and wherein a condenser output stream 13 is introduced to a top section of distillation column 50. The condenser operates at a pressure of from 50 to 200 mbar, preferably from 100 to 130 mbar. The water rich top stream 14 may be purged or further separated and/or purified.

Distillation column 50 preferably comprises a reboiler 70 for reboiling at least part of the column bottom stream 7 and wherein a reboiler output stream 8 is introduced to a bottom section of distillation column 50. The reboiler output stream preferably comprises from 10 to 50 wt. %, preferably from 15 to 30 wt. % of vapor fraction.

The bottom stream 9 is introduced in heat recover unit 40 and may thereafter be recycled, at least partially, into reactor 10.

In accordance with the present invention it is preferred that only a single distillation column 50 is used for the removal of water.

The present inventors calculated that the use of heat recovery unit 40 allows for a reduction in heat duty for the reboiler 70 of about 35 to 45%, in particular for a 10 stage distillation column wherein the mother liquor is introduced in the distillation column at stage 7.

The invention further relates to a plant 100 for carrying out the method as disclosed herein, said plant comprising the several units as disclosed herein in the context of the method.

More in particular the invention accordingly relates to a plant 100 comprising
at least one reactor (10) for carrying out a reaction between phenol and acetone in the presence of an ion-exchange resin catalyst, at least one crystallisation unit (20) in fluid communication with said reactor (10) for crystallising bisphenol A and/or bisphenol A/phenol adduct crystals, at least one solid-liquid separation unit (30) in fluid communication with said crystallisation unit (20) for separating bisphenol A or bisphenol A/phenol adduct crystals from a mother liquor,
a heat recovery unit (40) in fluid communication with said solid-liquid separation unit (30) and receiving said mother liquor, a distillation column (50) in fluid communication with said heat recovery unit (40), wherein said distillation column (50) comprises means for removing a bottom stream, said means being in fluid communication with said heat recovery unit (40) for exchanging heat between said bottom stream and said mother liquor.

Reactor 10 can be an upflow or a downflow reactor as described herein. Reactor 10 comprises means for feeding of raw materials such as acetone and phenol. Typically such means includes piping, pumps, valves and the like as is well known to the skilled person.

Reactor 10 is in fluid communication with at least one crystallisation unit 20, which means product stream 3 coming from reactor 10 can be fed to said crystallisation unit 20. In the crystallisation unit(s) 20 bisphenol A crystals or bisphenol A/phenol adduct crystals are formed, which are contained in the mother liquor as a slurry 4.

Slurry 4 is fed from crystallisation unit 20 into at least one solid-liquid separation unit 30, which accordingly is in fluid communication with said crystallisation unit 20. In the solid-liquid separation unit 30 the slurry is separated into a stream with solid bisphenol A or bisphenol A/phenol adduct and a mother liquor stream 5. The stream with solid bisphenol A or bisphenol A/phenol adduct can be worked-up in one or more further units so as to provide pure bisphenol A. Such units are well known to the skilled person.

Mother liquor 5 is fed from solid-liquid separation unit 30 to the heat recovery unit 40 which accordingly is in fluid communication with said solid-liquid separation unit 30. As explained the heat recovery unit 40 allows heat to be exchanged between the mother liquor 5 and a bottom stream 9 coming from a distillation column 50 located downstream, and in fluid communication with heat recovery unit 40. The cooled bottom stream 11 leaving heat recovery unit 40 can be fed to reactor 10 using any suitable means known to the skilled person, typically comprising piping and one or more pumps.

The plant 100 comprises a distillation column 50 in fluid communication with said heat recovery unit 40 for purifying the mother liquor and separating the mother liquor into a water-rich top stream 12 and a phenol-rich bottom stream 7. The distillation column preferably comprises a reboiler for reboiling at least part of the phenol rich bottom stream 7. Likewise the distillation column preferably comprises a condenser for condensing at least part of the column top stream (12). The use of reboilers and condensers in distillation columns is well known to the skilled person.

The method and apparatus as disclosed herein are described on the basis of a single unit for each of the reactor 10, the crystallisation unit 20, the solid-liquid separation unit 30, the heat recovery unit 40 and the distillation column 50. However, the present invention is not limited with respect to the amounts of each of those units and their mode of operation.

Accordingly a plant 100 may comprise a number of production lines with each line comprising one or more reactors 10 that can be operated in parallel and optionally in an alternating manner allowing maintenance without disrupting the continuous operation and further providing flexibility in terms of production volume.

By way of example a number of reactors 10 may be operated in parallel and product streams coming from each of the reactors 10 may be combined in one or more product streams that are consecutively fed into one or more crystallisation units 20. That is, the plant 100 may comprise one or more crystallisation units 20 operating in parallel. Each crystallisation unit 20 in turn may comprise one or more crystallisers operating in series for obtaining high purity crystals. Likewise each crystallisation unit 20 may comprise parallel lines of one or more crystallisers as also explained above in the context of the method.

On a similar token one or more solid liquid separation units 30 may be operated in parallel following a crystallisation unit 20.

If applicable, the mother liquor 5 coming from operating solid-liquid separation units may be combined and fed to heat recovery unit 40 followed by distillation in distillation column 50. Preferably the number of distillation columns is limited. It is furthermore preferred that the method or the plant 100 according to the invention does not comprise two or more distillation columns for the purification of the mother liquor operating in series.

The effect of the feed location of the (heated) mother liquor stream 6 into column 50 was studied using known modeling tools. The distillation column 50 was designed to have 10 equilibrium stages. The condenser 60 operated at 130 mbar pressure and the reboiler 70, configured as a thermosiphon reboiler, was operated such that the reboiler output stream 8 contained a vapor fraction of 25 wt. %. The mother liquor stream 6 contained 77.1 wt. % of phenol, 2.9 wt. % of water, 0.36 wt. % of acetone, 10.6 wt. % of p,p-BPA, 4.0 wt. % of o,p-BPA and several impurities in minor amounts including chroman, BPX-I, BPX-II, dimers. The phenol rich bottom stream comprised 79.3 wt. % of phenol and 0.1 wt. % of water whereas the water rich top stream comprised 91.0 wt. % of water and 6.5 wt. % of phenol.

The table below shows the effect of the feed stage on the reboiler Heat Duty, the condenser heat duty and the reflux ratio.

| | Reboiler | Condenser | |
|---|---|---|---|
| Feed | Heat | Heat | Reflux |
| Stage | Duty(MW) | Duty(MW) | Ratio |
| 5^{th} | 9.95 | −8.32 | 2.33 |
| 6^{th} | 6.2 | −4.64 | 0.86 |
| 7^{th} | 5.08 | −3.53 | 0.42 |
| 8^{th} | 5.26 | −3.72 | 0.48 |

As shown in the table, the feed stage was varied from 5th to 8th stage and the corresponding reboiler heat duty, condenser heat duty & reflux ratio were calculated. Surprisingly it was found that introducing the feed on 7th stage is more beneficial to reduce energy consumption of the distillation column for this feed.

1. Method for the continuous manufacture of bisphenol A comprising
feeding one or more feed streams comprising phenol and acetone to a reactor and reacting said acetone and phenol in the presence of an ion-exchange resin catalyst thereby forming a product stream comprising bisphenol A, phenol, acetone, water and by-products,
crystallising bisphenol A and/or bisphenol A/phenol adduct crystals from said product stream in one or more crystallisation units thereby forming a slurry consisting of said crystals and a mother liquor,
separating the mother liquor from the crystals in one or more solid-liquid separation units,
heating the mother liquor in a heat recovery unit and feeding the heated mother liquor to a distillation column,
separating the mother liquor in the distillation column thereby forming a bottom stream comprising phenol and optionally a minor amount of water and a top stream comprising water and optionally a minor amount of phenol, and
cooling the bottom stream in said heat recovery unit,
wherein in the heat recovery unit heat is exchanged between the bottom stream and the mother liquor liquor

2. The method of claim 1 wherein the heat recovery unit comprises one or more heating and/or cooling units which rely on external energy for heating or cooling.

3. The method of claim 1 wherein the bottom stream comprises from 75 to 85 wt. % of phenol and from 0 to 0.3 wt. % of water, based on the weight of the bottom stream.

4. The method of claim 1 wherein the top stream comprises from 5 to 12 wt. % of phenol, from 92 to 99 wt. % of water and optionally from 2 to 3 wt. % of acetone based on the weight of the top stream.

5. The method of claim 1 wherein during the separating of the mother liquor from the crystals in one or more solid-liquid separation units additional phenol is added to the mother liquor.

6. The method of 1 wherein the bottom stream leaving the heat recovery unit is fed as a feed stream to the reactor.

7. The method of 1 wherein the distillation for separating of the mother liquor is performed in a single distillation column.

8. The method of claim 7 wherein distillation column comprises n stages and wherein the mother liquor is introduced between stage n-5 and n-2, wherein n is from 7 to 13.

9. The method of claim 7 wherein the distillation column comprises a condenser for condensing at least part of the top stream and wherein a condenser output stream is introduced to a top section of the distillation column, wherein said condenser operates at a pressure of from 50 to 200 mbar.

10. The method of claim 8 wherein the distillation column comprises a reboiler for reboiling at least part of the bottom stream and wherein a reboiler output stream is introduced to a bottom section of the distillation column, wherein said reboiler output stream comprises from 10 to 50 wt. %.

11. The method of claim 1 wherein the ion-exchange catalyst comprises an attached promotor for increasing the catalyst selectivity.

12. The method of claim 1 wherein the product stream comprises
from 10 to 40 bisphenol A,
from 55 to 75 wt. % phenol,
from 0 to 3 wt. % of acetone,
from 2 to 20 wt. % by-products,
from 0 to 5 wt. % water.

13. Plant for carrying out the method of claim 1 comprising
at least one reactor for carrying out a reaction between phenol and acetone in the presence of an ion-exchange resin catalyst,
means for feeding phenol and/or acetone to said reactor,
at least one crystallisation unit in fluid communication with said reactor for crystallising bisphenol A and/or bisphenol A/phenol adduct crystals,
at least one solid-liquid separation unit in fluid communication with said crystallisation unit for separating bisphenol A or bisphenol A/phenol adduct crystals from a mother liquor,
a heat recovery unit heating in fluid communication with said solid-liquid separation unit and receiving said mother liquor,
a distillation column in fluid communication with said heat recovery unit, and
means for removing a top stream from said distillation column,
wherein said distillation column comprises means for removing a bottom stream, said means being in fluid communication with said heat recovery unit for exchanging heat between said bottom stream and said mother liquor.

14. Plant of claim 13 further comprising one or more of
means for feeding the bottom stream coming from the heat recovery unit to said reactor,
a reboiler for reboiling at least part of the column bottom stream,
a condenser for condensing at least part of the column top stream.

15. The method of claim 8 wherein distillation column comprises n stages and wherein the mother liquor is introduced between stage n-5 and n-2, wherein n is from 9 to 11.

16. The method of claim 9 wherein said condenser operates at a pressure of from 100 to 130 mbar.

17. The method of claim 11 wherein no separate sulfur containing promotor is added to the reactor.

18. The method of claim 12 wherein the product stream comprises
from 15 to 35 wt. % bisphenol A,
from 60 to 70 wt. % phenol,
from 0.01 to 1.0 wt. % of acetone,
from 5 to 15 wt. % by-products, and
from 1 to 3 wt. % water.

19. The method of claim 10 wherein said reboiler output stream comprises from 15 to 30 wt. % vapor fraction.

20. The method of claim 18 wherein the product stream comprises
from 20 to 30 wt. % bisphenol A.
